(19) United States
(12) Patent Application Publication
(10) Pub. No.: US 2006/0004424 A1
(43) Pub. Date: Jan. 5, 2006

(54) Charge-metered biomedical stimulator

(75) Inventors: Gerald E. Loeb, South Pasadena, CA (US); Jack D. Wills, Los Angeles, CA (US)

Correspondence Address: MCDERMOTT WILL & EMERY LLP, Suite 3400, 2049 Century Park East, Los Angeles, CA, 90067, US

(73) Assignee: University of Southern California

(21) Appl. No.: 11/146,761

(22) Filed: Jun. 6, 2005

Related U.S. Application Data
(60) Provisional application No. 60/577,440, filed on Jun. 4, 2004.

(51) Int. Cl. A61N 1/18

(52) U.S. Cl. 607/63; 607/7; 607/11

(57) ABSTRACT

Disclosed are biomedical stimulators and systems that deliver stimulus power efficiently to electrodes and tissues, provide reliable control of stimulus efficacy over a wide dynamic range of available power and voltage, avoid damaging net direct current flow through tissue, minimize the amount of data that must be transmitted to specify a particular stimulus strength, and extend the range of received field strengths for which stimulators can function safely and reliably. These biomedical stimulators and systems provide reliable stimulation of known intensity by measuring charging currents and discharging predetermined quantities of charge.

### CROSS-REFERENCE TO RELATED APPLICATIONS

This application claims priority to U.S. Provisional Patent Application Ser. No. 60/577,440 filed Jun. 4, 2004, entitled “Charge-Metered Biomedical Stimulator,” the entire contents of which are incorporated herein by reference.

### STATEMENT OF GOVERNMENT RIGHTS

The present invention has been made under a grant of NSF, federal grant no. EEC-0310723. Accordingly, the government may have certain rights to the present invention.

### BACKGROUND

1. Field

This application relates generally to devices and methods for electrical stimulation of biological tissues.

2. General Background and State of the Art

Power management can be important in the successful operation of electronics implanted in humans. In order to ensure the safety and effectiveness of biomedical stimulators, biomedical stimulators are typically designed to produce output pulses that are regulated on the basis of their pulse duration and either their voltage or current. The electronic circuitry for a voltage-regulated pulse reduces the operating voltage of the circuitry to the desired voltage, dissipating the excess energy as heat. The electronic circuitry for a current-regulated pulse continuously adjusts the output voltage so as to maintain a constant flow of current through the electrodes, dissipating the excess energy as heat.

Typically, for general use with unspecified physiological requirements or electrodes, it is desirable to have current-regulated stimulation with a wide range of finely controlled steps of current and pulse duration and as high a compliance voltage as feasible. Various existing biomedical stimulators often have circuits that require substantial space for current regulating transistors. Such circuits tend to go out of current regulation if the product of the requested current and the impedance of the rest of the circuit, including electrodes, leads and perhaps coupling capacitors, exceeds the available compliance voltage. This problem can be particularly severe for stimulators powered by inductive coupling, whose operating voltages tend to fluctuate with the strength of that coupling. The likelihood of this happening can be reduced by operating the circuitry at a range of voltages much higher than actually required for the stimulus currents anticipated (i.e. providing “head room” in the compliance voltage), but this can greatly increase the amount of electrical energy that is dissipated as heat by the circuitry rather than delivered to the tissue itself. In applications requiring large numbers of densely packed stimulation channels, such heating can damage surrounding tissues.

Typical circuits in voltage-regulated biomedical stimulators also drop most of the supply voltage across the output transistors so as to accommodate a wide range of possible output voltage levels, resulting in very low efficiency unless the requested output voltage is quite close to the supply rail voltage. As a consequence high efficiency operation is typically difficult to achieve.

Stimulation of excitable tissues such as neurons and muscle cells typically depends on the integration of charge by their cell membranes. This integration may occur efficiently over a fairly wide range of stimulus pulse widths that depends on the membrane time constant of the stimulated cell(s). For example, most myelinated nerve axons have a membrane time constant around 100 μs, so they can be efficiently stimulated with pulses whose durations range from about 30-300 μs. The effective strength of the stimulation pulse will be the charge delivered by the pulse, which is the integral of the current flow during the stimulation pulse (i.e. the product of stimulus current and pulse duration for so-called “square” pulses with regulated current output).

During biomedical stimulation, one should typically avoid net direct current flow through electrodes and tissues. Direct current (DC) can result in irreversible electrochemical reactions between the electrodes and body fluids that produce damaging corrosion and electrolysis products. This may be avoided by employing biphasic pulses in which the charge delivered by the stimulating pulse is followed by equal charge delivered in the opposite direction before the next stimulation pulse is delivered. This can be accomplished asymmetrically by employing a capacitor in series with the electrode and then discharging this capacitor through the electrodes between pulses or by making the electrode itself function as an electrolytic capacitor (a so-called “capacitor electrode” as described previously in the art (U.S. Pat. No. 5,312,439, incorporated herein by reference). However, the required capacitor tends to be physically bulky; the time to discharge it fully may be substantial and the voltage that accumulates on it during the stimulation pulse reduces the head-room of the compliance voltage. The reverse pulse can be delivered explicitly by a current-regulated pulse that is equal and opposite to the stimulation pulse. However, this requires active electronic circuitry operating with the opposite voltage and matched to that responsible for the stimulation pulse, which is difficult to guarantee, especially if the power supply voltages are fluctuating.

### SUMMARY

The exemplary embodiments of the charge meter circuits, systems and methods described herein can be used to control power and stimulation in biomedical stimulators. They can avoid all of the above shortcomings by directly controlling the single stimulus parameter that determines both the safety and efficacy of a stimulation pulse, namely its charge. The power-efficient design can compensate for fluctuations and nonlinearities of electrode and contact impedance, and can reduce or eliminate residual post-stimulation charge to extend electrode life and minimize tissue damage.

In one aspect of the biomedical stimulation, a method for stimulating tissue comprises delivering a voltage potential across a tissue, measuring the amount of charge flowing through the tissue as a consequence of the voltage potential, and removing the voltage potential from the tissue when the amount of measured charge has reached a predetermined value. Optionally and advantageously, the same charge measuring circuitry can be used to control the delivery of an equal and opposite charge to the electrodes and tissue.

In another aspect of the biomedical stimulation, a method for stimulating tissue comprises delivering charge into a charge storage device that is in-series with the tissue to be stimulated; measuring the amount of charge that is being delivered to the charge storage device; terminating the delivery of charge to the charge storage device when the amount of charge delivered to the charge storage device reaches a predetermined value; and delivering the stored charge into tissue.

In yet another aspect of the biomedical stimulation, a method for stimulating tissue comprises storing energy in an energy-storage device between stimulation pulses; using the stored energy to energize a stimulation pulse; measuring the amount of charge that is being delivered during the stimulation pulse; and terminating the delivery of charge to the tissue when the amount of charge delivered reaches a predetermined value.

It is understood that other embodiments of the devices and methods will become readily apparent to those skilled in the art from the following detailed description, wherein it is shown and described only exemplary embodiments of the devices, methods and systems by way of illustration. As will be realized, the devices, systems and methods are capable of other and different embodiments and its several details are capable of modification in various other respects, all without departing from the spirit and scope of the invention. Accordingly, the drawings and detailed description are to be regarded as illustrative in nature and not as restrictive.

### BRIEF DESCRIPTION OF THE DRAWINGS

Aspects of the biomedical stimulation devices and systems are illustrated by way of example, and not by way of limitation, in the accompanying drawings, wherein:

FIGS. 1A-1B include a block diagram schematically illustrating a monophasic, capacitor-coupled charge-metered stimulation system and its associated output waveforms of voltage and current;

FIGS. 1C-1D include a block diagram schematically illustrating a monophasic, capacitor-powered charge-metered stimulation system and its associated output waveforms of voltage and current;

FIGS. 1E-1F include a block diagram schematically illustrating a biphasic charge-metered stimulation system and its associated output waveforms of voltage and current;

FIG. 2 illustrates an exemplary charge meter circuit;

FIG. 3 illustrates an exemplary current sense circuit with a differential amplifier;

FIG. 4 illustrates a circuit with a gated integrator, comparator, and DAC;

FIG. 5 illustrates several exemplary waveforms relating to the charge metering circuit of FIGS. 3 and 4;

FIG. 6 is an illustration of the electrical circuit of a microstimulator; and

FIG. 7 is a block diagram of the electronic control for an implanted biomedical stimulator.

### DETAILED DESCRIPTION

The detailed description set forth below in connection with the appended drawings is intended as a description of exemplary embodiments and is not intended to represent the only embodiments in which the biomedical stimulation devices, methods and systems can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the biomedical stimulation devices, methods and systems. However, it will be apparent to those skilled in the art that the biomedical stimulation devices, methods and systems may be practiced without these specific details.

Effective stimulus of nerve tissue can benefit from monitoring and controlling the amount of charge delivered, rather than monitoring and controlling pulse duration or current. For example, during a stimulus pulse produced by discharging a capacitor electrode, charge could be metered by measuring the discharge current flowing through the electrodes and integrating until it reaches a specified value, whereupon the discharge could be stopped and a capacitor can be recharged to the compliance voltage. Pulse current and duration would then be a byproduct of compliance voltage and electrode impedance rather than controlled variables. If compliance voltage or electrode impedance fluctuated, the requested charge would still be delivered as a result of automatic off-setting changes in pulse current and duration. Operating range could be controlled by having a few values of regulated compliance voltage, each of which would tend to produce a different current depending on the impedance of the electrodes. Little or no power would be dissipated by the stimulus control circuitry because it could simply provide a very low resistance path for current to flow through the electrodes while discharging the capacitor electrode. The current flow through said stimulus control circuitry could be integrated and monitored by the charge-metering circuitry described below, regardless of the particular compliance voltage at which the output is being energized.

One example of an implantable biomedical stimulator which may benefit from charge-regulated stimulus control is the BION™ (BIONic Neurons; Alfred E. Mann Institute, University of Southern California). BIONs™ are a new class of implantable medical device: separately addressable, single channel, electronic microstimulators (16 mm long×2 mm in diameter), that can be injected in or near muscles and nerves to treat paralysis, spasticity and other neurological dysfunctions. Microstimulators that may be used in various embodiments are described in U.S. Pat. Nos. 5,193,539; 5,193,540; 5,312,439; and 5,324,316, each of which are incorporated by reference in their entirety. A BION typically may include a tantalum electrode at one end and an iridium electrode at the opposite end. Each BION™ may receive power and digital command data by a radio frequency electromagnetic field to produce functional or therapeutic electrical stimulation. For use in this invention, the electrodes may be configured for selective interaction with the surfaces of an injection device, including but not limited to the cannula lumen or probe distal end for example. Capacitive, power-storing electrodes can be kept charged to the regulated compliance voltage by the recharge current, but the actual voltage available on them at any instant typically depends on the charge removed by the previous stimulation pulse(s) and the duration and current level of the intervening recharge phase(s). The recharge current may be kept at a sufficiently low level that it does not by itself cause stimulation, particularly when the implant is first powered-up and the capacitor electrode is charged from zero.

Another example of an implantable biomedical stimulator which may benefit from charge-regulated stimulus control is a multichannel retinal prosthesis, in which large numbers of closely spaced electrodes can be stimulated in complex temporospatial patterns, as described in U.S. Pat. Nos. 5,109,844, 5,935,155, 6,393,327, and 6,718,209, which are incorporated herein by reference. Because of the severe size limitations on the implanted electrode array and stimulus generation circuitry, such biomedical stimulators could benefit from charge-metering that could minimize wasted electrical power, heat dissipation and physical size of the electrode contacts and electronic circuitry. Electrodes are typically made from noble metals such as platinum and iridium. Coupling capacitors can be impractical, so charge-balancing to avoid net DC could be accomplished reliably by the electronic circuitry itself. The electrical power to such circuitry is typically provided by inductive coupling of an internal receiving coil to a transmission coil outside the body. This coupling is subject to fluctuations due to relative motion between the external and internal coils, which may produce fluctuations in the available compliance voltages for driving current in either direction through the electrodes.

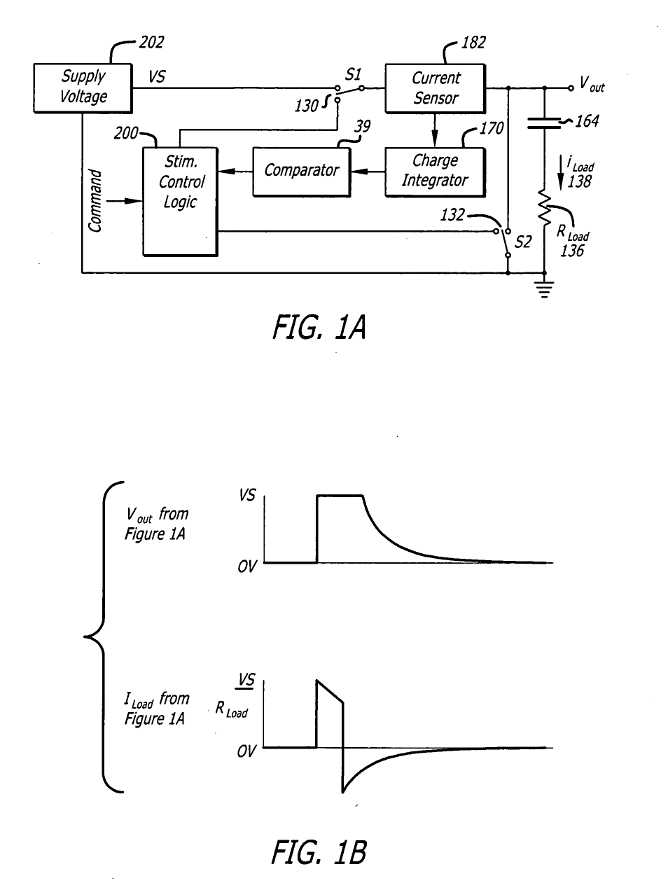

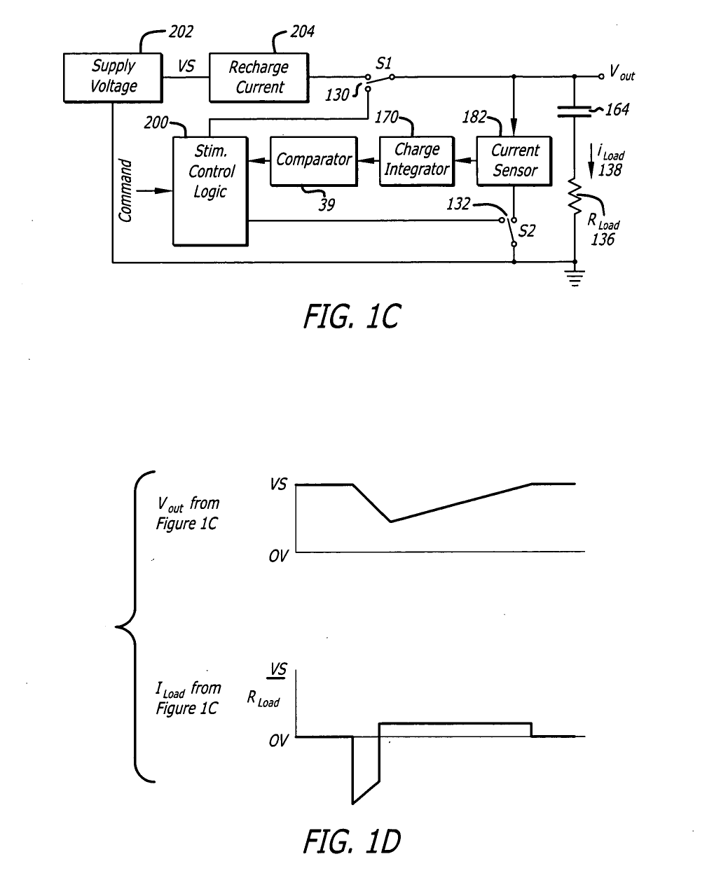

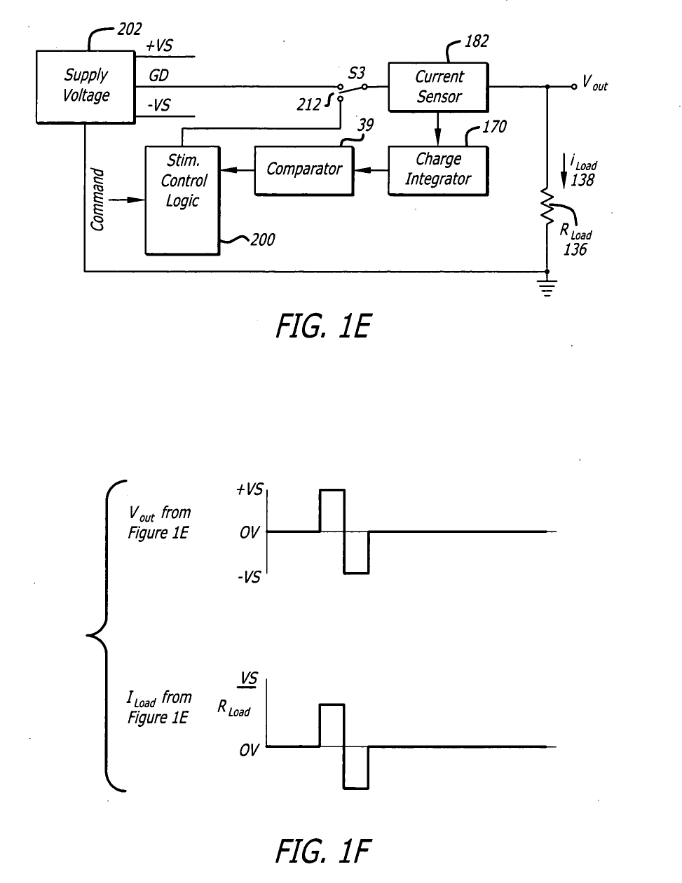

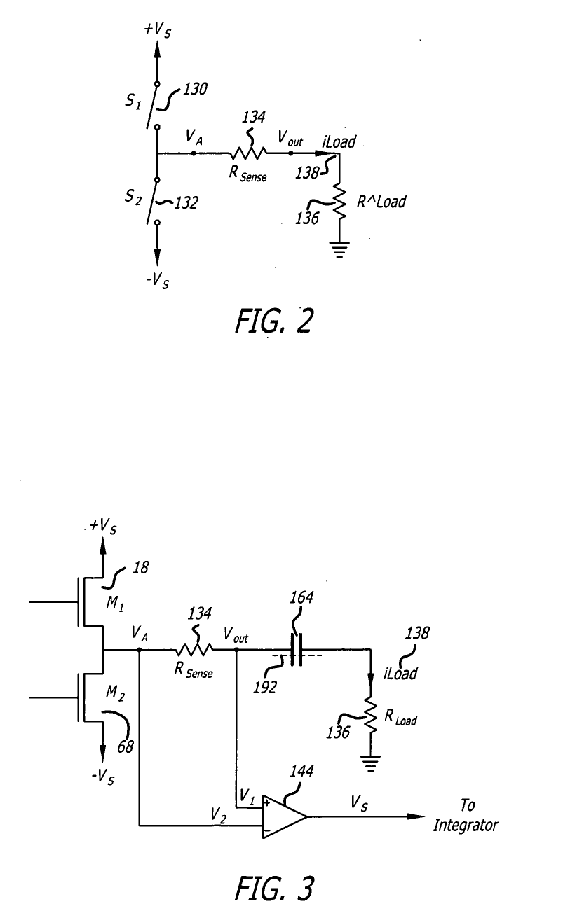

Referring to the schematic block diagram in FIG. 1A, one example of a charge-metering system comprises a current controller that provides current to an output circuit that includes an energy storage device; and a current sensor that is used to determine the current going to an energy storage apparatus. In FIG. 1A, the energy storage device is a capacitor 164, which may be either a discrete electronic component or the capacitance of the electrode itself in contact with the body fluids. Resistor R_{LOAD} 136 designates the combined impedance of the electrode interface with the tissue and the tissue itself, which is generally a complex, nonlinear impedance. The capacitor 164 can be in-series with the tissue to be stimulated so that charge flowing into the capacitor constitutes a current I_{LOAD} 138 passing through the tissue. Following a command to the stimulus control logic 200, switch S1 130 can be closed and switch S2 132 can be opened, so that supply voltage 202 (Vs) can energize the output circuit resulting in current I_{LOAD} 138. The current sensor 182 can be used by the charge measuring device (which may include a differential amplifier 144 and integrator 170, as illustrated in FIGS. 2 and 3 and discussed below) to determine the amount of charge that is delivered through the circuit. The information from the charge measurement can be fed to the comparator 39, which can then compare the charge to the charge specified as part of the command information. When the correct amount of charge has passed through the tissue, the stimulus control logic 200 can open S1 130 and close S2 132, allowing charge that has accumulated in capacitor 164 to discharge through the electrodes and tissue, achieving the desired charge balance. This mode of operation may be identified as monophasic, capacitor coupled because only the first phase of the stimulus waveform is directly controlled. FIG. 1B shows voltage and current waveforms that may result from the system described in FIG. 1A.

FIG. 1C illustrates another example of a charge meter system. This system can be useful when the supply voltage source 202 does not provide sufficient power to create the desired stimulus pulse during the pulse itself, such as in some inductively-powered BION microstimulators. The limited recharge current 204 that can be produced by supply voltage 202 (Vs) can be applied continuously between stimulation commands by keeping S1 130 closed and S2 132 open, causing capacitor 164 to charge to Vs, whereupon current ceases to flow in the output circuit. Capacitor 164 can be either a discrete electronic component or the capacitance of the electrode itself in contact with the body fluids. In response to a stimulation command, the stimulus control logic can open S1 130 and close S2 132. The voltage Vs stored on capacitor 164 can cause current I_{LOAD} 138 to flow through the body tissue R_{LOAD} 136 and through the current sensor 182, which can be in series. The current sensor 182 can be used by the charge measuring device (which may include a differential amplifier 144 and integrator 170, as illustrated in FIGS. 2 and 3 and discussed below) to determine the amount of charge that is delivered through the circuit. The information from the charge measurement can be fed to the comparator 39, which can then compare the charge to the charge specified as part of the command information. When the correct amount of charge has passed through the tissue, the stimulus control logic 200 can open S2 132 and close S1 130. The recharge current 204 can again flow into capacitor 164 through R_{LOAD} 136 until the voltage across capacitor 164 equals Vs, achieving the desired charge balance between the two phases of the stimulus pulse. This mode of operation can be identified as monophasic, capacitor powered because only the first phase of the stimulus waveform is directly controlled but the power for the stimlus pulse comes from energy stored previously on capacitor 164. FIG. 1D shows voltage and current waveforms that may result from the system described in FIG. 1C.

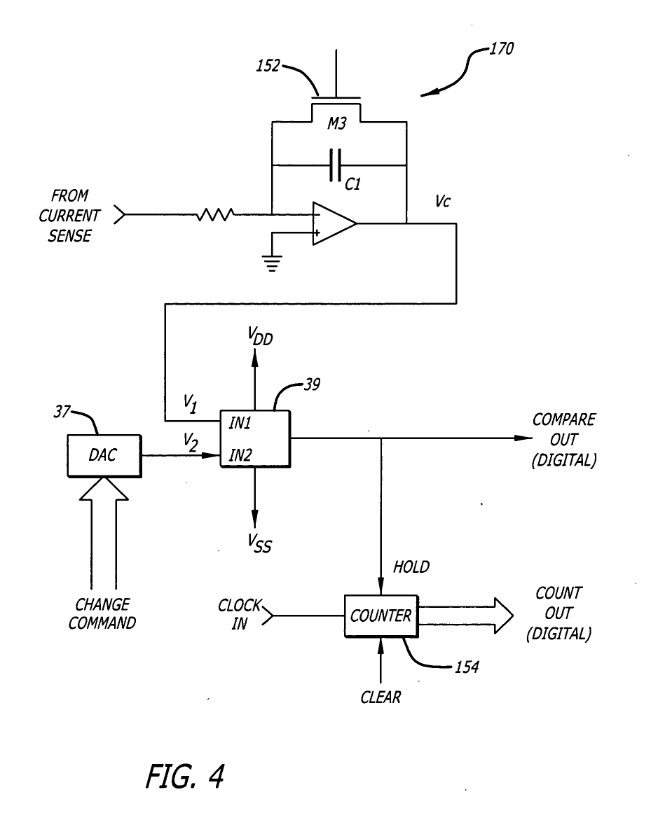

FIG. 1E illustrates a charge meter stimulus control system that can provide biphasic stimulation, in which each phase of the stimulation pulse can be explicitly controlled. Advantageously, a capacitor is not required to achieve charge-balance. This may be useful for dense multichannel systems such as a retinal prosthesis where it could be difficult to provide a capacitor for each output channel. When stimulation is not required, switch S3 212 can be connected to supply voltage ground (Gd). No current flows through R_{LOAD} 136. When a stimulus command is received, stimulus control logic 200 can switch S3 212 for one of the two available output voltages, +Vs or −Vs. For the output waveforms illustrated in FIG. 1F, S3 212 is switched initially to +Vs, which can cause the first phase of current I_{LOAD} 138 to flow through the current sensor 182 and R_{LOAD} 136 (the electrodes and tissue). The current sensor 182 can be used by the charge measuring device (which may include a differential amplifier 144 and integrator 170, as illustrated in FIGS. 2 and 3 and discussed below) to determine the amount of charge that is delivered through the circuit. The information from the charge measurement can be fed to the comparator 39, which then compares the charge to the charge specified as part of the command information. When the correct amount of charge has passed through the tissue, the stimulus control logic 200 can switch S3 212 to the opposite polarity supply voltage, here illustrated as −Vs. This can cause the opposite polarity of current to flow in the output circuit, generating the second phase of stimulus current I_{LOAD} 138 as illustrated. As described below and illustrated in FIG. 4, the charge integrator can be operated so as to determine exactly when the amount of charge that has flowed in the second phase of stimulation is equal and opposite to that which flowed during the first phase. At that point, the stimulus control logic 200 can switch S3 212 to ground, causing the stimulation to cease. Any residual charge that might have accumulated on the electrodes through slight errors in the charge measurements will be discharged during the interval between successive stimulation commands. FIG. 1F shows voltage and current waveforms that may result from the system described in FIG. 1E.

In the charge meter systems described in FIGS. 1A-F, the supply voltages Vs may be fixed or programmable according to other commands and control circuitry not illustrated but known to those skilled in the art. By selecting a different supply voltage, the operator can change the range of currents that would actually flow through the tissue. This may be advantageous in order to ensure that the stimulus pulses actually delivered have durations that lie within the range for which the structure to be excited tends to integrate charge to reach threshold.

FIG. 2 illustrates a basic charge-meter circuit used in a configuration similar to FIG. 1E, in which R_{SENSE} 134 represents a linear sense resistor with a low value of resistance (much less than R_{LOAD} 136 ) that can be incorporated within the stimulus control and generation circuitry and R_{LOAD} 136 represents the impedance of the excitation probe in place (such probes may include, for example, resistive or non-polarizing electrodes or capacitor electrodes or other charge-delivery or charge storage apparatuses known to those skilled in the art). R_{LOAD} 136 which is generally a complex, nonlinear impedance. To obtain high efficiency, transistors can be used as switches 130 and 132 that are either on or off. Rather than set the output voltage V_{OUT} to some particular value, the entire rail voltage (+Vs or −Vs) is applied to R_{LOAD} 136 and the amount of time T that the switch S₁ 130 is closed is used to control the amount of charge delivered to R_{LOAD} 136. The charge meter measures the charge delivered to the load and then turn off S₁ 130 when it reaches a predetermined charge amount. The charge can then be drained by reversing the transistor switch settings until charge balance is achieved. Because the probe and tissue act as a nonlinear, time varying resistance, the two output transistors may be alternately turned on for differing amounts of time to achieve charge balance.

For example, in the exemplary circuit illustrated in FIG. 2,

$${i}_{LOAD} = \frac{{V}_{A} - {V}_{out}}{{R}_{SENSE}}$$

Since R_{LOAD} 136 is nonlinear and time varying, i_{LOAD} 138, the current of the load, will not be constant. However, as R_{SENSE} 134 is a linear on chip resistor:

$${i}_{LOAD} ( t ) = \frac{{V}_{A} ( t ) - {V}_{out} ( t )}{{R}_{SENSE}}$$

$$and$$

$${Q}_{LOAD} = {\int }_{0}^{T} {i}_{LOAD} ⅆ t = \frac{1}{{R}_{SENSE}} {\int }_{0}^{T} {V}_{A} ( t ) - {V}_{OUT} ( t ) ⅆ t$$

While operating the circuit, at first the switch S₁ 130 is closed and kept closed until time T when:

$${Q}_{LOAD} = \frac{1}{{R}_{SENSE}} {\int }_{0}^{T} {V}_{A} ( t ) - {V}_{OUT} ( t ) ⅆ t$$

$$or$$

$${\int }_{0}^{T} {V}_{A} ( t ) - {V}_{out} ( t ) ⅆ t = {R}_{SENSE} {Q}_{LOAD}$$

This gives the first half of a biphasic waveform. The second half of a biphasic waveform can be created by opening S₁ 130 and closing S₂ 132. Again, I_{LOAD} 138 can be integrated to obtain the correct amount of (dis)charge. Given the nonlinear, time varying nature of R_{LOAD} 136, the charge and discharge phases are expected to take differing amounts of time.

FIG. 3 illustrates a current-sensing circuit that can be used to measure the current, and consequently the charge, delivered to the capacitor 164 and to load R_{LOAD} 136. In some embodiments, the capacitor is removed from the circuit and replaced by a short 192. In such embodiments, there is no charge storage and the charge from the power supply is sufficient for stimulation. The output stage comprises two CMOS switch transistors M1 18 and M2 68, a sense resistor 134, and a unity gain difference amplifier 144. The output voltage V_{B} of the difference amplifier 144 measures the instantaneous output load current (V_{OUT}=V₁−V₂).
V_{B}=Vₒᵤₜ−V_{A}=−i_{LOAD}R_{SENSE}

FIG. 4 illustrates a circuit with a gated integrator and DAC 37, which receives the current from the current sense circuit of FIG. 3. The output voltage of the comparator 39 is V_{SS} if V₁>V₂; and V_{DD}if V₁ <V₂. (It may be advantageous to operate the comparator 39 and digital logic 37 at voltages different from those used to energize the electrodes, herein designated as +Vs and −Vs.) The output voltage (V_{C}) of the operational integrator is:

$${V}_{out} = {V}_{initial} - \frac{1}{{R}_{1} {C}_{1}} {\int }_{0}^{T} {V}_{in} ( t ) ⅆ t$$

The following example demonstrates how a charge meter circuit may be operated.

The CMOS switches 18 and 68 illustrated in FIG. 3 are controlled by two digital signals:
Mode High→current flows in R_{LOAD} Low→open circuit-no current
Polarity High→current flows into R_{LOAD} Low→current flows out of R_{LOAD}

This can be expressed by the truth table:

| Mode | Polarity | Gate M1 | Gate M2 |
|---|---|---|---|
| 0 | 0 | 1 | 0 |
| 0 | 1 | 1 | 0 |
| 1 | 0 | 1 | 1 |
| 1 | 1 | 0 | 0 |

First, under initial conditions the MODE is set to low, the POLARITY is high, the M3 CMOS switch transistor 152 is closed, and the counter 154 is off. The desired charge is then selected. Specifically, a digital code is loaded into the DAC 37, thus setting the amount of charge delivered:

$${Q}_{LOAD} = {V}_{DAC} \frac{{R}_{1} {C}_{1}}{{R}_{SENSE}}$$

When a trigger pulse is received, the MODE is set to High (which closes M1 in FIG. 3). M3 is simultaneously opened (enabling the integrator), and the counter is started. Current then flows into R_{LOAD} (in FIG. 3). The output V_{B} of the difference amplifier 144 measures the instantaneous current i_{LOAD}. The integrator output voltage V_{C} is a measure of the charge delivered. The proper amount of charge has been delivered when V_{C} reaches V_{DAC} (and the comparator output switches).

By adding counter 154, it is possible to measure the mean output impedance of the probe R_{LOAD}, which may be dominated by the electrodes and tissue Specifically, counter 154 can be started when the first switch is closed to energize the output circuit and stopped at the moment the comparator output switches state. The time duration shown by the counter 154 is a measure of the average resistance of the probe:

$${R}_{LOAD}^{MEAN} = \frac{{V}_{DD}}{{V}_{DAC}} {R}_{SENSE} \frac{T}{{R}_{1} {C}_{1}}$$

After the first phase of stimulation, the load can be discharged to achieve charge balance. Specifically, the POLARITY is set to low, which opens M1 18, closes M2 68, and begins to discharge the load. The DAC 37 is also reset to output zero volts. The integrator output voltage V_{C} will continually decrease as R_{LOAD} is discharged until V_{C} reaches zero. When V_{C} reaches zero, the comparator output switches. This causes the MODE signal to go High, which opens M1 18 and M2 68.

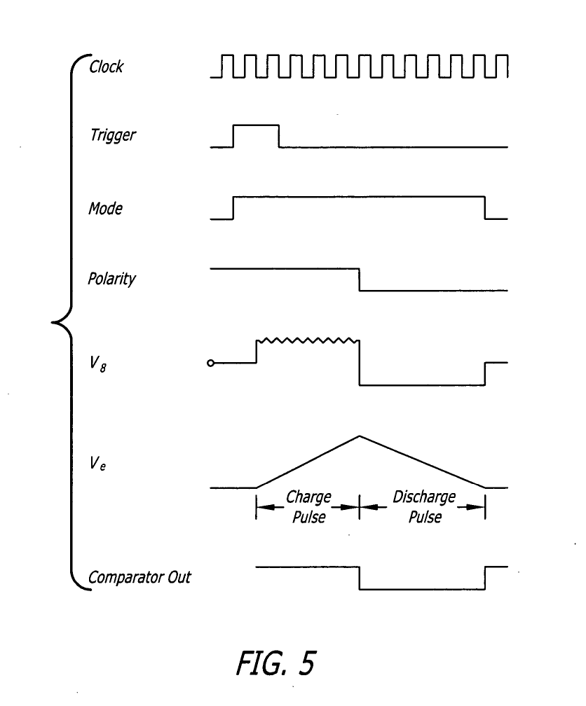

Finally, the circuit is reset. M3 152 is turned on so that the integrating capacitor C₁ is discharged. The POLARITY is set to High and ready for the next charge command and trigger pulse. An additional switch (not illustrated) may be employed to connect the output to ground, as illustrated in FIG. 1E. A set of waveforms corresponding to the charge meter circuit is illustrated in FIG. 5.

The charge meter circuitry can be used to control stimulus intensity in a variety of biomedical stimulators. For example, in an implantable microstimulator such as the BION, stimulus pulse strength could be defined and commanded in units of charge (e.g., nC). Commanded stimulus charge values could cover a wide dynamic range from 40-20,000 nC with an exponential series whose resolution could be 3-10% at any value. Compliance voltage could be settable in coarse steps from the lowest value required to operate the logic (˜3V) to the higher value available from the foundry process (˜24V). One reasonable series could be 3, 6, 12 and 24 VDC. The source of power may arise from inductive coupling, battery, or other form known to those skilled in the art. Biomedical stimulators having a charge meter can be used to produce stimuli with varying waveforms, such as monophasic and biphasic for example. If +Vs and −Vs used to energize the electrodes are not equal and opposite, then the currents flowing during successive phases of a biphasic stimulation pulse will be unequal, which may be useful for specialized applications such as anodal block and others known to electrophysiologists.

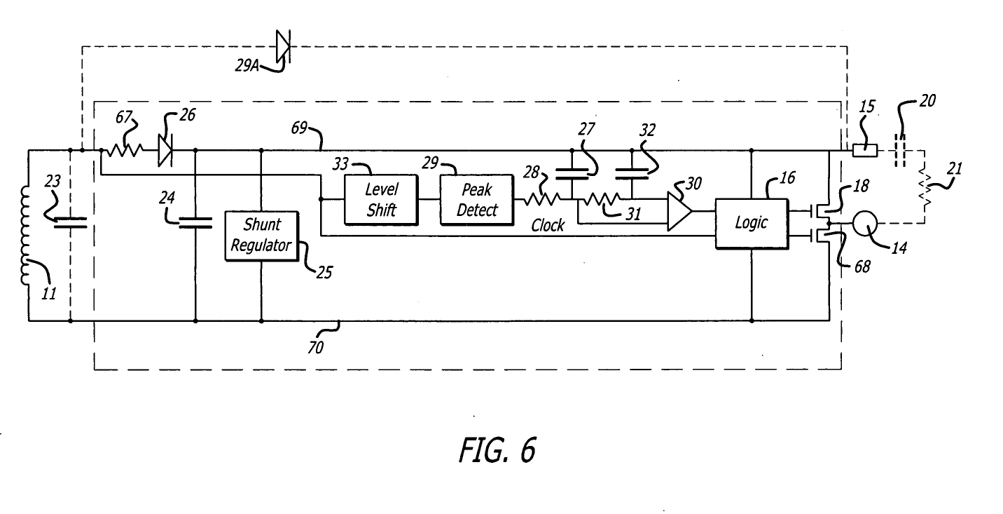

FIG. 6 is an illustration of the electrical circuit of a microstimulator such as the BION, which operates in the monophasic, capacitor powered mode illustrated in FIG. 1C. Most of the electrical circuit of the microstimulator is contained on an integrated circuit, or microcircuit, chip 22. The coil 11 is tuned by capacitor 23 to the frequency of the alternating magnetic field. In some instances, capacitor 23 may be provided by the stray capacitance of coil 11. Resistor 67 and Schottky diode 26 provide rectification and a power bus 69 for the positive side of the received electrical energy. If it is desired, an external diode, such as that shown at 26A may be utilized. It is connected around microcircuit chip 22, from one end of coil 11 to the external connection of the electrode 15. This external diode 26A is particularly useful in the event the chip diode 26 fails or does not meet the product specification or would otherwise prevent the electronic chip 22 from being usable or acceptable. Capacitor 24 serves to smooth out the ripple in the power bus 69. Shunt regulator 25 serves as a current shunt to prevent the voltage between the positive and negative power busses 69 and 70 (and thus between the electrodes 15 and 14) from becoming too high, say, above 15 volts. Shunt regulator 25 may be comprised of one or more Zener diodes and resistors or more sophisticated voltage regulating circuitry. The shunt regulator 25 effectively controls the excess energy which is received by dissipating it at an acceptable rate. It is expected that dissipation would not exceed approximately 4 milliwatts/cm², which is about 20% of levels which have been found acceptable in cardiac pacemaker dissipation.

It is pointed out that lowering the Q of the power supply and the receiving circuit, by a shunt-regulator which dissipates energy or provides a current-sinking path, effectively stabilizes the electronic control circuit, particularly the demodulator and detector so that variations in loading do not interfere with signal demodulation or detection. At the same time, the shunt-regulator, or current-sinking means, prevents overcharging or overloading the storage capacitor means in the microstimulator.

Level shift 33 is connected to receive the energy received by the receiving coil 11 and drops the peaks to a detection range so the peak detector 29 can detect the peaks. From that detected signal, a short term detected signal is obtained by capacitor 27 and resistor 28 and a long term average detected signal is obtained by capacitor 32 and resistor 31 (which have a longer time constant than the first resistor and capacitor). The short term detected signal and the long term average detected signal are fed into comparator 30 which provides the detected data to be processed by the logic 16. Such logic controls the stimulation transistor 18 and the recharge transistor 68. When transistor 18 is conducting, transistor 68 is non-conducting and the current flow between electrodes 14 and 15 is used to provide a stimulating pulse. In the preferred embodiment only a small part of the charge stored in the capacitance of the electrodes is utilized in the stimulating pulse.

Logic 16, would not require the full voltage of the V+ between lines 69 and 70, and may be operated on 2 to 4 volts, by a series regulator, (not shown) which would reduce and control the supply voltage to logic 16.

In order to restore the full charge between electrodes 14 and 15, or, in other words, the charge on the capacitor 20, transistor 18 is controlled to be non-conducting and transistor 68 is controlled to be conducting and the voltage busses 69 and 70 charge up the electrodes.

If the microstimulator does not use anodized, porous tantalum or other structure which provides an electrolytic capacitor when disposed in the body fluids, then a miniature capacitor may be required to be disposed inside the housing of the microstimulator. Such capacitor may be manufactured on the electronic chip 22, but is preferably external to the electronic chip 22. An electrolytic capacitor 82, having 1-10 microfarads, would be typical. The required value depends on the maximal charge to be delivered in a single stimulus pulse and the amount of recharge current available between stimulus pulses.

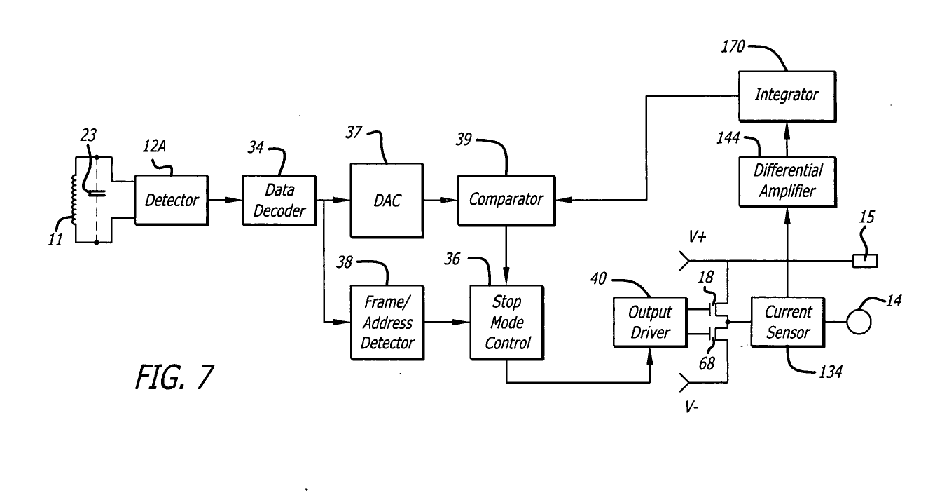

FIG. 7 is a block diagram illustrating one example of the circuitry, including charge-metering, of an electronic control means of a BION microstimulator. Assuming a 2 MHz, modulated, alternating magnetic field is transmitted from outside the skin, coil 11 and capacitor 23 provide the signal at that frequency to data detector 12A. Assuming that the modulating information is contained in 36-bit frames, data detector 12A provides such 36-bit frame data to data decoder 34.

Data decoder 34 sends the data, to DAC 37 and the frame/address detector 38. DAC 37 is essentially a CMOS RAM storage device which stores only a portion of the received frame, in this instance, amount of desired charge.

Frame/address detector 38 looks at an incoming frame bit by bit and determines whether the information is addressed to this microstimulator. It also checks the validity of the entire frame, which may be parity-encoded to insure accuracy. In the preferred embodiment, Manchester encoding of the bit transmission is also used.

The mode control 36 calls for one or the other of two modes, one mode, “generate pulse” and the other mode, “search for valid frame”. If a valid 36-bit frame is received by detector 38, it notifies mode control 40 which switches to “generate pulse” mode. The output driver 40 controls transistor 18 which is turned on to allow a stimulating pulse for the requisite time as determined when comparator 39 determines that the charge, from the integrator 170 (in connection with sense resistor 134 and differential amplifier 144 described in FIG. 3 and FIG. 4), is equal to the desired charge value stored in DAC 37. When such counts are equal, comparator 39 advises mode control 36 (that the desired charge has been reached) and to stop. Mode control 36 then stops driver 40 which turns off transistor 18, so that it is non-conducting. While transistor 18 is turned on, of course, tantalum electrode 15 and iridium electrode 14 are discharging a portion of the electrical charge between them, which is stored on capacitor 20, FIG. 6, which is an integral part of anodized tantalum electrode 15, thus providing a stimulating pulse through the body.

Transistor 68 is controlled by output driver 40 to restore the full charge on anodized tantalum electrode 15 with respect to iridium electrode 14, in preparation for the next stimulating pulse. The recharge current could be 100 microamps, in high recharge, and 10 microamps, in low recharge. Commanded stimulus charge values could cover a wide dynamic range from 40-20,000 nC with an exponential series whose resolution could be 3-10% at any value.

The charge meter may require fewer bits of command data to achieve a given resolution of stimulus strength than would be required by conventional stimulators. While conventional stimulators typically require data explicitly specifying both the stimulus amplitude (voltage or current) and pulse duration, stimulators using the present charge meters may function with just specification of stimulus charge. This may be advantageous when many stimulation commands must be transmitted at a high rate via a channel with limited bandwidth, such as via telemetry.

The charge meter stimulator may also avoid the dissipation of power in voltage or current control circuits that have substantial resistance compared to that of the electrodes and tissues through which the stimulus must flow. This is because the switches used to energize the output in charge meter stimulators do not have to perform an amplitude control function, and thus may be operated in a low resistance mode to during the generation of a stimulus waveform. This may be advantageous if power conservation is important, as in battery powered devices, or if heating of the implanted device is a concern, as in physically small, multichannel stimulators.

The previous description of the disclosed embodiments is provided to enable any person skilled in the art to make or use the microstimulator injection devices, methods and systems. Various modifications to these embodiments will be readily apparent to those skilled in the art, and the generic principles defined herein may be applied to other embodiments without departing from the spirit or scope of the devices, methods and systems described herein. Thus, the charge meter circuits, devices, methods and systems are not intended to be limited to the embodiments shown herein but are to be accorded the widest scope consistent with the principles and novel features disclosed herein.

What is claimed is:

1. A method for stimulating tissue, comprising:
a) delivering a voltage potential across a tissue;
b) measuring the amount of charge flowing through the tissue as a consequence of the voltage potential; and
c) removing the voltage potential from across the tissue when the amount of measured charge has reached a predetermined value.

2. The method of claim 1, wherein the measuring of the charge flowing through the tissue includes integrating a current signal.

3. The method of claim 1, further comprising delivering an opposite voltage potential across a tissue.

4. A method for stimulating tissue, comprising:
d) delivering charge into a charge storage device;
e) measuring the amount of charge that is being delivered to the charge storage device;
f) terminating the delivery of charge to the charge storage device when the amount of charge delivered to the charge storage device reaches a predetermined value; and
g) delivering the stored charge into tissue.

5. The method of claim 4, wherein the charge storage device is in series with the tissue to be stimulated.

6. The method of claim 4, wherein the measuring of the charge that is being delivered to the charge storage device includes integrating a current signal.

7. The method of claim 4, wherein the delivering the stored charge into the tissue comprises shorting the charge storage device.

8. A method for stimulating tissue, comprising:
a) delivering charge into a charge storage device;
b) delivering a stimulation pulse to tissue that is energized by the stored charge;
c) measuring the amount of charge that is being delivered during the stimulation pulse;
d) terminating the delivery of charge to the tissue when the amount of charge delivered reaches a predetermined value.

9. The method of claim 8, wherein the charge storage device stores charge between stimulation pulses.

10. The method of claim 8, wherein the charge storage device is in series with the tissue to be stimulated.

11. The method of claim 8, wherein the measuring of the charge that is being delivered to the charge storage device includes integrating a current signal.

12. The method of claim 8, wherein the delivering the stored charge into the tissue comprises shorting the charge storage device.
